(19) United States
(12) Patent Application Publication
(10) Pub. No.: US 2010/0291689 A1
(43) Pub. Date: Nov. 18, 2010

(54) Lanthanide Complexes as Fluorescent Indicators for Neutral Sugars and Cancer Diagnosis

(75) Inventors: Robert M. Strongin, Portland, OR (US); Onur Alptürk, Baton Rouge, LA (US); Oleksandr Rusin, Portland, OR (US); Sayo O. Fakayode, Baton Rouge, LA (US); Jorge O. Escobedo Cordova, Portland, OR (US); Weihua Wang, Baton Rouge, LA (US); Isiah M. Warner, Baton Rouge, LA (US); William E. Crowe, Baton Rouge, LA (US); Vladimir Král, Prague (CZ)

Correspondence Address: PATENT DEPARTMENT;TAYLOR, PORTER, BROOKS & PHILLIPS, L.L.P, P.O. BOX 2471, BATON ROUGE, LA, 70821-2471, US

(73) Assignees: BOARD OF SUPERVISORS OF LOUISIANA STATE UNIVERSITY & AGRICULTURAL AND MECHANICAL COLLEGE, Baton Rouge, LA (US); INSTITUTE OF CHEMICAL TECHNOLOGY, Prague 6 (CZ)

(21) Appl. No.: 12/293,483

(22) PCT Filed: Mar. 14, 2007
(86) PCT No.: PCT/US2007/063943

§ 371 Date: Aug. 3, 2010

Related U.S. Application Data
(60) Provisional application No. 60/784,733, filed on Mar. 21, 2006.

(51) Int. Cl. G01N 33/48; G01N 33/92; G01N 33/574; C07F 5/00; C07C 251/00

(52) U.S. Cl. 436/64; 436/71; 436/94; 534/15; 556/33; 564/248

(57) ABSTRACT

A group of water-soluble salophene-lanthanide complexes and other salophene-metal complexes are useful for purposes including: (i) detecting neutral carbohydrates at physiologically-relevant pH, (ii) the selective detection of gangliosides, and (iii) the selective detection of lysophosphatidic acid (LPA) in the presence of phosphatidic acid. The selective detection of LPA is useful in diagnosing ovarian and other cancers.

(In countries other than the United States:) The benefit of the 21 Mar. 2006 filing date of United States provisional patent application 60/784,733 is claimed under applicable treaties and conventions. (In the United States:) The benefit of the 21 Mar. 2006 filing date of U.S. provisional patent application 60/784,733 is claimed under 35 U.S.C. §119(e).

### FILE NO. STRONGIN 0521W

The development of this invention was partially funded by the United States Government under Grant R01 EB002044 awarded by the National Institutes of Health. The Government has certain rights in this invention.

### TECHNICAL FIELD

This invention pertains to the detection of neutral sugars and to the diagnosis of cancers in biological samples, by fluorescent detection with lanthanide complexes or other metal complexes.

### BACKGROUND ART

In nature, saccharides are recognized by lectins. An important mode of lectin binding involves the coordination of a carbohydrate ligand to a metal center. C-type binding lectins recognize saccharides in a calcium-dependent manner.

There is an unfilled need for sugar indicators that function efficiently under neutral-pH, physiologically relevant conditions. A major problem in the detection of neutral sugars with artificial receptors has been competitive binding by bulk water.

There is an unfilled need for the improved detection of sialic acid-containing gangliosides. An increase or decrease in total sialic acid levels (conjugated plus freely circulating) in biological fluids is diagnostic for certain cancers. But there are no existing methods for detecting sialic acid that are well-suited for clinical diagnosis. Prior methods for detecting sialic acid have included the acid-catalyzed liberation of bound sialic acid residues from gangliosides, followed by assay for sialic acid. This method typically results in destruction of the analyte, lowering the accuracy of the assay by decreasing the amount of material available for measurement. Enzymatic hydrolysis can result in incomplete sialic acid liberation, limiting accurate analysis. There have also been some approaches using metal-based sugar indicators at high pH.

Y. Ci et al, Anal. Chem., vol. 67, pp. 1785-1788 (1995) disclose that DNA may be selectively monitored with a europium(III)-tetracycline (Eu—Tc) complex in the presence of RNA, via fluorescence monitoring at the europium emission wavelength of 615 nm. The Eu—Tc complex exhibits fluorescence emission enhancement upon complexation via displacement of bound water. However, the Eu—Tc complex is not selective, and also exhibits fluorescence emission enhancement in the presence of several neutral sugars and anions.

F. van Veggel et al., “Metallomacrocycles: Supramolecular chemistry with hard and soft metal cations in action,” Chem. Rev., vol. 94, pp. 279-299 (1994) provides a review of the chemistry of weak interactions (hydrogen bonds, ion-dipole, dipole-dipole, van der Waals, etc.) of metallomacrocycles that contain combinations of hard and soft metal cations, the latter category including transition metal cations.

S. Striegler et al., “A sugar discriminating binuclear copper(II) complex,” J. Am. Chem. Soc., vol. 125, pp. 11518-11524 (2003) discloses a binuclear copper complex that was found to differentiate between D-mannose and D-glucose at high pH, as measured by UV-Vis absorption.

A. Davis at al., “Carbohydrate recognition through noncovalent interactions: A challenge for biomimetic and supramolecular chemistry,” Angew. Chem. Int. Ed., vol. 38, pp. 2978-2996 (1999) is a review of the contemporaneous state of the art in carbohydrate recognition. The review noted that carbohydrate recognition remained a challenge to supramolecular chemists, and that the principals of saccharide recognition by biomolecules were not well understood, and it described some of the progress that had been made.

J. Bruce et al., “The selectivity of reversible oxy-anion binding in aqueous solution at a chiral europium and terbium center: Signaling of carbonate chelation by changes in the form and circular polarization of luminescence emission,” J. Am. Chem. Soc., vol. 122, pp. 9674-9684 (2000) discloses reversible anion binding in aqueous media at chiral Eu(III) and Tb(III) as measured by ¹H NMR and by changes in the emission intensity and circular polarization with an alkylphenanthridinium chromophore. Using a series of heptadentate tri-amide or polycarboxylate ligands, the affinity for carbonate/bicarbonate, phosphate, lactate, citrate, acetate, and malonate at pH 7.4 was found to decrease as a function of the overall negative charge on the complex, with malonate binding most strongly.

L. Sillerud et al., “Assignment of the ¹³C nuclear magnetic resonance spectrum of aqueous ganglioside G_{M1} micelles,” Biochemistry, vol. 17, pp. 2619-2628 (1978) discloses the ¹³C NMR spectrum of ganglioside GM1 from beef brain, and spectral perturbations induced by paramagnetic europium(III).

Millions of women are at high risk for ovarian cancer. Some 26,000 new cases are diagnosed each year in the United States alone. There is an unfilled need for more effective methods for the early diagnosis of ovarian cancer. One of the factors that makes ovarian cancer so dangerous is that it is very difficult to detect early enough to allow effective treatment. Survival rates improve dramatically when the disease is discovered while the cancer is still localized in the ovaries. Methods currently used to detect ovarian cancer include ultrasound, laparoscopy, and positron emission tomography. While sonography shows promise for early detection, it is too expensive to use for widespread, routine screening.

Plasma lysophosphatidic acid (LPA) levels are an important marker for ovarian cancer, and possibly other gynecological cancers. LPA differs from the more common phosphatidic acid (PA) in having only one fatty acid residue per lipid molecule. LPA could provide a useful diagnostic marker for ovarian and other gynecological cancers if there were a reliable method of determining LPA that could readily be implemented in a clinical setting. One study reported a concentration range for LPA in plasma in healthy controls from below 0.1 to 6.3 μM, with a mean of 0.6 μM; while the concentration in patients with ovarian cancer was between 1 and 43.1 μM, with a mean of 8.6 μM. See Y. Xu et al., “Lysophosphatidic Acid as a Potential Biomarker for Ovarian and Other Gynecologic Cancers,” JAMA, vol. 280, pp. 719-723 (1998). However, the analytical method used by Y. Xu et al. for detecting LPA is too lengthy and complex for routine clinical use. Briefly, the Xu et al. method employed lipid extraction; separation of LPA from other lipids on thin-layer chromatographic plates; developing with a solvent system of chloroform-methanol-ammonium hydroxide; scraping sample spots from the silica gel plates into glass centrifuge tubes; hydrolysis in ethanolic potassium hydroxide; transmethylation in the presence of behenic acid (internal standard) with boric chloride-methanol; extracting fatty acid methyl esters with petroleum ether; drying under nitrogen; re-dissolving in chloroform; and analysis by gas chromatography.

### DISCLOSURE OF THE INVENTION

We have discovered that a group of water-soluble salophene-lanthanide complexes and other salophene-metal complexes are useful for several purposes, including: (i) detecting neutral carbohydrates at physiologically-relevant pH, (ii) the selective detection of gangliosides, and (iii) the selective detection of lysophosphatidic acid (LPA) in the presence of phosphatidic acid (PA). The selective detection of LPA is useful in diagnosing ovarian and other gynecological cancers. A number of the salophene-lanthanide complexes and other salophene-metal complexes are themselves believed to be novel compositions of matter.

A salophene is a condensation product of an ortho-hydroxyl aldehyde and an aromatic amine. Typical novel salophene-lanthanide complexes in accordance with the present invention, Compounds 1 and 2, are depicted below:

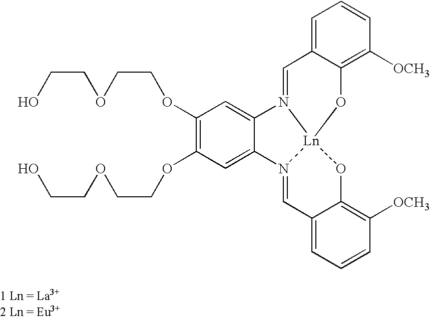

Another lanthanide (Ln) may also be used to form a homologous compound: In addition to La and Eu, the lanthanides also include Ce, Pr, Nd, Pm, Sm, Gd, Tb, Dy, Ho, Er, Tm, Yb, and Lu.

Alternatively, an actinide may be used in the compounds of this invention: Ac, Th, Pa, U, Np, Pu, Am, Cm, Bk, Cf, Es, Fm, Md, No, or Lr. Many of the actinides are radioactive. In some settings radioactivity would be a disadvantage, but in other applications radioactivity can be an advantage, as it provides an alternative label to monitor a complex; and likewise for Ra or other radioactive elements or isotopes. As further alternatives, the other Group III B metals Sc and Y may be used in this invention, as may other transition metals: Ti, V, Cr, Mn, Fe, Co, Ni, Cu, Zn, Zr, Nb, Mo, Tc, Ru, Rh, Pd, Ag, Cd, Hf, Ta, W, Re, Os, Ir, Pt, Au, and Hg. As a further alternative, the Group II A metals Ca, Sr, Ba, and Ra may be used in this invention. Different metal atoms will impart differing selectivities.

The novel lanthanide-salophene complexes are generally water-soluble, and are useful, for example, in the fluorescence detection of carbohydrates and cancer biomarkers. The novel complexes may be used, for example, in the fluorescence detection of sialogangliosides without interference from asialogangliosides or sugar carboxylic acids. Additionally, we have selectively detected lysophosphatidic acid in the presence of phosphatidic acid, a measurement that can be useful in the diagnosis of ovarian and other gynecological cancers.

The observed fluorescence changes are those associated with the ligand(s) coordinated to the metal atom. The fluorescence of the ligand(s) is altered as a result of binding to a target molecule. While our observations to date have been that fluorescence is generally enhanced as the result of binding to a target molecule, in some cases fluorescence may instead be reduced. Either increased or decreased fluorescence may be used in detection, so long as fluorescence is altered as a result of binding to a target molecule.

The lanthanide complexes are useful in detecting neutral sugars as well as glyco- and phospholipids. In solutions at physiological pH, the fluorescent lanthanide complexes can bind neutral sugars with apparent binding constants comparable to those of arylboronic acids. Interference from common anions is minimal. For example, the europium complex (Compound 2) successfully detected sialic acid-containing gangliosides at pH 7.0 in the presence of an asialoganglioside. This selectivity is attributed, at least in part, to cooperative complexation of the oligosaccharide and sialic acid residues to the metal center. In methanol (MeOH) solution, lysophosphatidic acid (LPA), a biomarker for several pathological conditions including ovarian cancer, has been selectively detected using Compound 2. We have successfully detected LPA in spiked human plasma samples by fluorescence monitoring. The 2-sn-OH moiety of LPA may play an important role in binding to the metal center. We have found that other molecules found in common brain ganglioside and phospholipid extracts did not interfere with the ganglioside or LPA fluorescence assays.

Lanthanides and calcium share some similar properties, despite their differing valences. Trivalent lanthanides (e.g., La³⁺, Eu³⁺), actinides, and Ca²⁺ exhibit a strong affinity for saccharides as compared to most other metal ions. Interestingly, lanthanides can extend their ligand coordination number by the addition of either neutral or charged ligands through ligand-sphere extension, leading to highly coordinated complexes.

The present invention overcomes prior obstacles in detecting neutral sugars with artificial receptors. Compound 1, for example, mimics the calcium-saccharide interactions of C-type lectins, and allows for the successful detection of neutral mono- and oligosaccharides in neutral buffer solution. As another example, Compound 2 exhibited enhanced fluorescence emission with anionic lipid analytes with proximal hard atom (e.g., oxygen) coordination sites, such as the alpha hydroxyl of LPA or the oligosaccharide hydroxyls of gangliosides. Compound 2 may be used, for example to selectively detect (i) sialic acid-containing gangliosides in buffer solution, or (ii) LPA, for example LPA in MeOH. The latter, in particular, is useful in diagnosing ovarian cancer and other gynecological cancers.

Ionic interactions predominate in lanthanide coordination chemistry. Eu³⁺, which has a smaller ionic radius than La³⁺, should exhibit a higher affinity towards anionic substrates. More generally, a smaller ionic radius in a lanthanide should strengthen intramolecular ligand interactions.

Compound 2 is also useful in recognizing charged glycolipids. Glycolipids contain multiple potential sites for interactions with both the metal center and the ligand-binding sites of Compound 2. The binding may readily be detected by fluorescence measurements. Compound 2 may be used, for example, in the selective detection of sialic acid-containing gangliosides.

### BRIEF DESCRIPTION OF THE DRAWINGS

FIG. 1 depicts relative fluorescence intensity changes for several saccharides mixed with Compound 1.

FIG. 2 depicts the relative fluorescence intensity changes of solutions of Compound 2 in the presence of various gangliosides, phospholipids (LPA and PA), and other charged and neutral analytes.

FIG. 3 depicts fluorescence emission for mixtures of Compound 1 with D-glucose at various concentrations.

FIG. 4 depicts the changes observed in relative fluorescence emissions for solutions containing various monosaccharides or oligosaccharides, anions, BSA, or a mixture of BSA and glucose, on the one hand; with Compound 1, on the other hand,

FIG. 5 depicts the structures of asialo-GM1 and GM1.

FIG. 6 depicts the coordination of GM1 to Eu³⁺, and also free sialic acid.

FIG. 7 depicts fluorescence intensity at various emission wavelengths for solutions of Compound 2, both alone and in combination with gangliosides

FIG. 8 depicts relative fluorescence intensities for solutions of compound 2 with various gangliosides, phospholipids, and other charged and neutral analytes.

FIG. 9 depicts the structures of disialogangliosides GD1a and GD1b.

FIG. 10 depicts fluorescence intensity versus wavelength for several Eu—Tc complexes with gangliosides or sialic acid

FIG. 11 depicts fluorescence intensity versus wavelength for Compound 2 and solutions of Compound 2 with either LPA or PA in MeOH.

FIG. 12 depicts relative fluorescence intensity changes for solutions of Compound 1 or 2 with LPA or PA in MeOH.

FIG. 13 depicts hypothesized intramolecular hydrogen bonding patterns of LPA and PA.

FIG. 14 depicts relative fluorescence intensity changes of solutions of Compound 2 in MeOH, in the presence of LPA, PA, or other charged and neutral analytes.

FIG. 15 depicts the structures of LPA and other phospholipids tested.

FIG. 16 depicts relative fluorescence emission versus concentration of LPA in methanolic extracts of blood plasma samples containing Compound 2.

FIG. 17 schematically depicts syntheses of Compounds 1 and 2.

FIGS. 18(a), (b), and 19 depict alternative ligands and structures.

FIGS. 20 and 21 depict relative fluorescence intensity changes for solutions of the structure shown in FIG. 19, wherein each R═H and Ln=Eu³⁺, with lysophospholipids in different solvents.

FIG. 22 depicts relative fluorescence intensity changes for solutions of the structure shown in FIG. 19, wherein each R═H and Ln=Eu³⁺ with phospho- and corresponding lysophospholipids in different solvents.

FIG. 23 depicts relative fluorescence intensity changes for solutions of the structure shown in FIG. 19, wherein each R═H and Ln=La³⁺ for sugar solutions containing various monosaccharides, oligosaccharides, anions, and BSA.

### MODES FOR CARRYING OUT THE INVENTION

### Example 1

Materials and Instrumentation. All reagents were purchased from Sigma-Aldrich, unless otherwise noted. Gangliosides were purchased from Calbiochem. Phospholipids were purchased from Avanti Polar Lipids. All reagents were used as purchased, without further purification, unless otherwise noted. Fluorescence spectra were recorded with a SPEX Fluorolog-3 spectrofluorimeter equipped with double excitation and emission monochromators, and a 400 W Xe lamp. ¹H and ¹³C NMR spectra were measured on a Bruker DPX-250 or DPX-300 spectrometer. All δ values are reported in ppm. Coupling constants are reported in Hz. Fourier-Transform Infrared spectra were measured on a Tensor 27 Infrared Spectrophotometer (Bruker Optics Inc.). Mass spectra were acquired on a Bruker ProFLEX III MALDI-TOF mass spectrometer.

### Example 2

Saccharide detection. Solutions of the saccharides, 1.1×10⁻³ M each, were prepared in HEPES buffer (0.1 M, pH 7.0). To the buffer solutions containing saccharides, Compound 1 was added to a final concentration of 5.53×10⁻⁶ M. Control solutions were prepared with only the HEPES buffer and Compound 1 at the same concentrations. All samples were incubated for 10 min at room temperature before fluorescence was measured.

### Examples 3 and 4

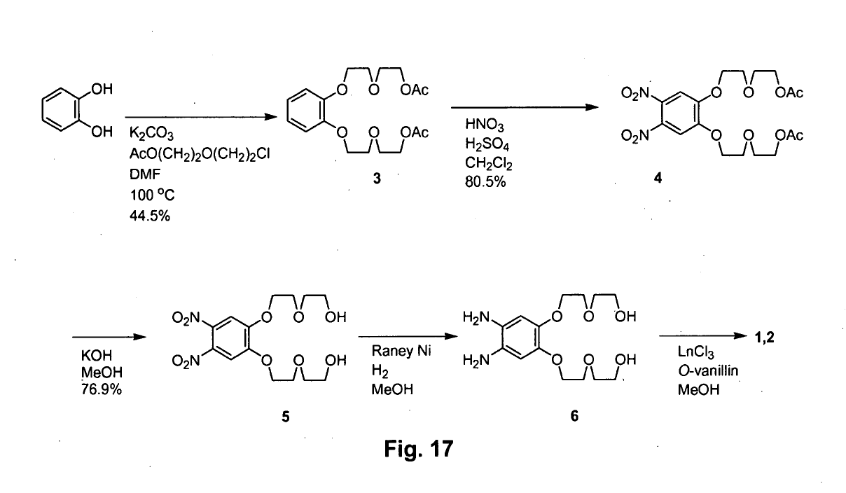

Syntheses of Compounds 1 and 2. The syntheses of Compounds 1 and 2 are depicted schematically in FIG. 17, and are described in greater detail below. Compounds 3, 4, 5, and 6 were reported in S. Duggan et al., J. Org. Chem., vol. 66, pp. 4419 ff (2001), while Compounds 1 and 2 are believed to be novel. In addition, each step of the synthesis shown in FIG. 17 is believed to be novel.

### Example 5

Compound 3, 1,2-bis(2-(2-(2-acetoxy(ethoxyethoxy))))benzene, was synthesized by adding catechol (1 g, 9.80 mmol) in DMF (20 mL), and O-acetyl-2-(2-chloro-ethoxy)-ethanol (2.1 g, 18.16 mmol) in DMF (10 mL), to a suspension of K₂CO₃ (3.76 g, 27.24 mmol) in DMF (60 mL) under N₂. This mixture was heated overnight at 100° C. Residual K₂CO₃ was then removed by filtration. The remaining reaction mixture was diluted with EtOAc (60 mL), and then washed with H₂O (4×30 mL). The organic phase was separated from the aqueous phase, after which the organic phase was dried over Na₂SO₄. The resulting material was concentrated under reduced pressure. The product was a yellow oil (1.5 g, 44.5%), which had the following characteristics: ¹H NMR (250 MHz, DMSO-d₆) δ (ppm): 1.99 (6H, s, CH₃), 3.69 (8H, m, CH₂), 4.09 (8H, m, CH₂), 6.92 (4H, m, ArH). ¹³C NMR (62.5 MHz, DMSO-d6) δ (ppm): 21.6, 65.0, 69.1, 69.3, 69.8, 115.2, 122.1, 149.2, 171.2.

### Examples 6 and 7

Compounds 4 and 5 were synthesized according to the procedures of S. Duggan et al., J. Org. Chem., vol. 66, pp. 4419 ff (2001). Spectroscopic data (¹H NMR and ¹³C NMR) were in agreement with the published data.

### Example 8

Compound 5 (0.2 g, 0.53 mmol) was dissolved in MeOH (15 mL). Raney nickel catalyst was added. Hydrogenation was then carried out at 50 psi and monitored via H₂ consumption. Residual Raney nickel was removed from the mixture by filtration through celite. The resulting Compound 6 is prone to oxidation, and was used immediately in the next step of the synthesis, without characterization, to reduce unwanted oxidation.

### Example 9

O-vanillin (0.16 g, 1.1 mmol) in 10 mL MeOH and the solution containing Compound 6 were concurrently added over 20 minutes to a refluxing solution of LaCl₃ (0.13 g, 0.53 mmol) in 10 mL MeOH. The solution was then refluxed for 2 hours. The reaction mixture was concentrated under reduced pressure, and the residue was washed 3 times with 5 mL EtOAc. The resulting product, Compound 1, was a dark-red solid (0.37 g) with the following characteristics: ¹³C NMR (62.5 MHz, DMSO-d₆) δ (ppm): showing peaks at 49.4, 56.5, 56.9, 61.1, 69.7, 69.8, 73.1, 73.3, 113.8, 114.0, 118.4, 120.0, 120.9, 123.4, 149.2, 151.4, 192.8. MALDI-Tof (m/z): calc'd. C₃₀H₃₄LaN₂O₁₀, 721.13; found, 721.48. IR (cm⁻¹): 3206.20, 1614.33, 1439.22, 1209.10, 1036.87.

### Example 10

Compound 2 was synthesized from Compound 6 as otherwise described above for Compound 1, except that EuCl₃ replaced the LaCl₃. The resulting product, Compound 2, was a dark-red solid (0.35 g) with the following characteristics: ¹³C NMR (62.5 MHz, DMSO-d₆) δ (ppm): 49.4, 56.6, 57.0, 61.1, 69.7, 69.8, 73.1, 73.4, 118.4, 119.3, 120.1, 120.9, 123.4, 147.3, 149.0, 149.3, 151.6, 192.8. MALDI-Tof (m/z): calc'd. C₃₀H₃₄EuN₂O₁₀, 735.14; found, 735.34. IR (cm⁻¹): 3104.00, 1638.44, 1444.54, 1214.76, 1018.07.

### Example 11

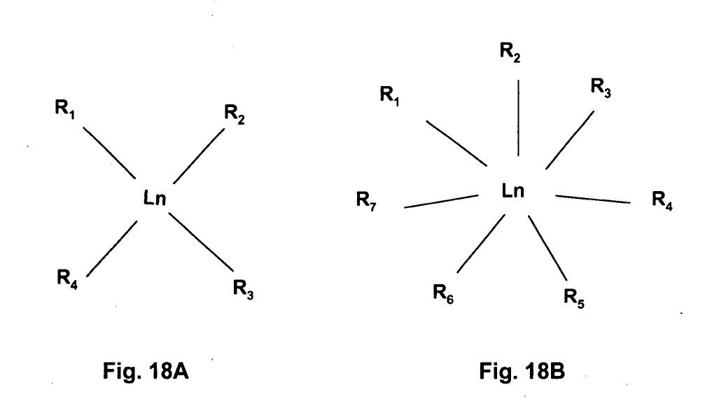

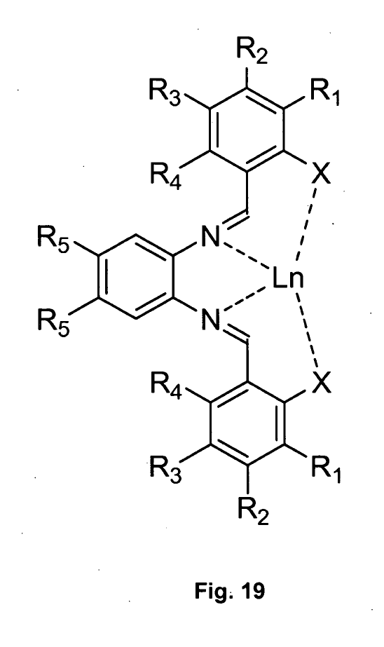

Analogs of Compounds 1 and 2 are prepared with other lanthanides, actinides, or other metals as previously described, but substituting the other corresponding metal chlorides in the step where the reaction occurs with Compound 6. More generally, other metal halides or metal salts may be used. Alternatively, other ligands or structures may be used, as depicted for example in FIGS. 18 and 19.

In FIGS. 18(a) and (b), Ln denotes a lanthanide, an actinide, a transition metal, Sc, Y, Ca, Sr, Ba, or Ra. The groups R₁, R₂, R₃, R₄, R₅, R₆, and R₇, denoting coordinating ligands (other than solvent molecules), may be the same or different. The preferred 4 ligands are depicted in FIG. 18(a). Compounds that may be used in the invention are not limited to those with 4 ligands, however. The invention may also be practiced with from 2 to 7 (non-solvent) coordinating ligands, as shown more generally in FIG. 18(b). In other words, from zero to five of the seven coordinating groups depicted in FIG. 18(b) may optionally be absent. The ligands may be the same or different. Some or all of the several ligands may optionally be covalently linked to one another.

The ligands may comprise one or more molecules per metal atom; i.e., both monodentate and polydentate ligands may be used. The ligand(s) (as a group) should possess the following characteristics; however, if multiple ligand molecules are used, it is not necessary that each ligand molecule must share each of these characteristics: There should be both polar and nonpolar groups, to promote binding to the polar and nonpolar regions of LPA (or other target). There should be aromatic rings. The aromatic rings serve multiple functions—they act as nonpolar groups, they engage in π-π interactions, and they alter fluorescence spectra. At least some of the ligand(s) should be water-soluble. There should be hard atoms (e.g., P, S, O, or N) available for coordinating to the metal atom. The ligands may, for example include halogen atoms, other heteroatoms (e.g., P, S, O, N), saturated or unsaturated C₁ to C₄ aliphatic chains, aromatic groups, glycol, polyethyleneglycol, phosphate, sulphate, and carboxylate.

In FIG. 19, Ln denotes a metal atom selected from the same group as listed above in connection with the compounds depicted in FIG. 18. R₁,R₂, R₃, R₄, and R₅ may be the same or different; and are independently selected from the group consisting of hydrogen, halogens, groups with heteroatoms (P, O, S, N), saturated or unsaturated C₁ to C4 aliphatic groups, aromatic groups, glycol, polyethyleneglycol, phosphate, sulphate, and carboxylate. The two X moieties may be the same or different; each X denotes a halogen atom (F, Cl, Br, I, At), a group V A nonmetal (N, P, As, Sb), or a group VI A nonmetal (O, S, Se, Te, Po).

### Example 12

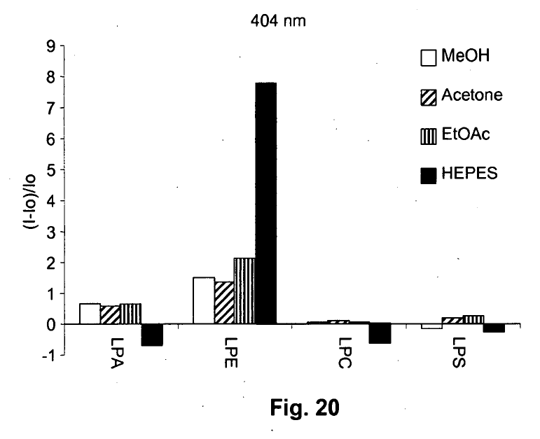

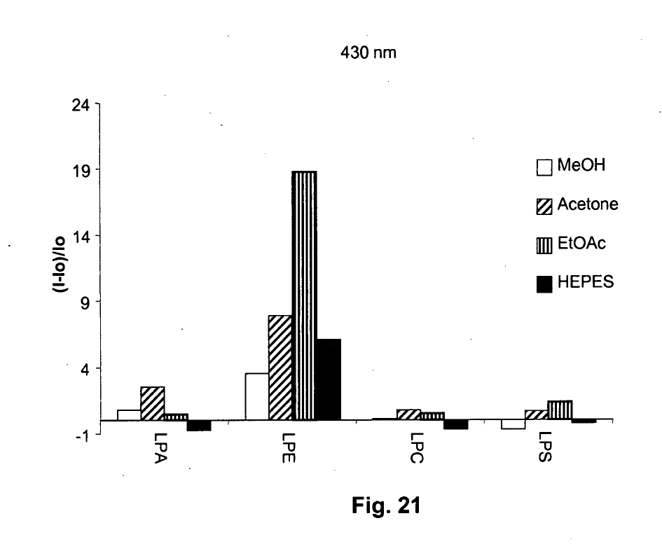

Altering the R-groups and metal atoms allows one to readily modify the selectivity of the complexes. FIG. 20 depicts relative fluorescence intensity changes for solutions of the structure shown in FIG. 19, wherein each R═H, Ln=Eu³⁺ (5.5×10⁻⁶ M), X═—OCH₃, with lysophospholipids (1×10⁻⁴ M) in different solvents. Excitation was at 360 nm, and emission was measured at 404 nm. LPA=lysophosphatidic acid; LPE=lysophosphatidyl ethanolamine; LPC=lysophosphatidyl choline; LPS=lysophosphatidyl serine. MeOH=methanol; EtOAc=ethyl acetate; HEPES=0.1 M HEPES pH 7.0. FIG. 21 depicts otherwise similar measurements, but with fluorescence emission measured at 430 nm.

### Example 13

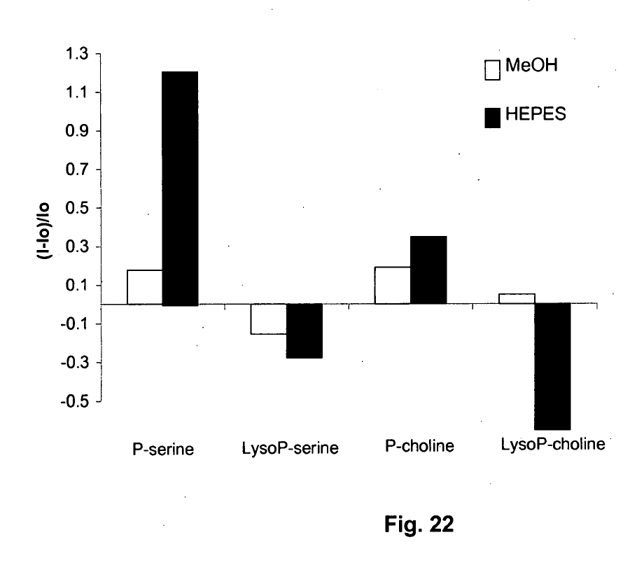

FIG. 22 depicts relative fluorescence intensity changes for solutions of the structure shown in FIG. 19, wherein each R═H, Ln=Eu³⁺ (5.5×10⁻⁶ M), and X═—OCH₃, with phospho- and corresponding lysophospholipids (1×10⁻⁴ M) in different solvents. Excitation was at 360 nm, and emission was measured at 404 nm. MeOH=methanol; HEPES=0.1 M HEPES pH 7.0.

### Example 14

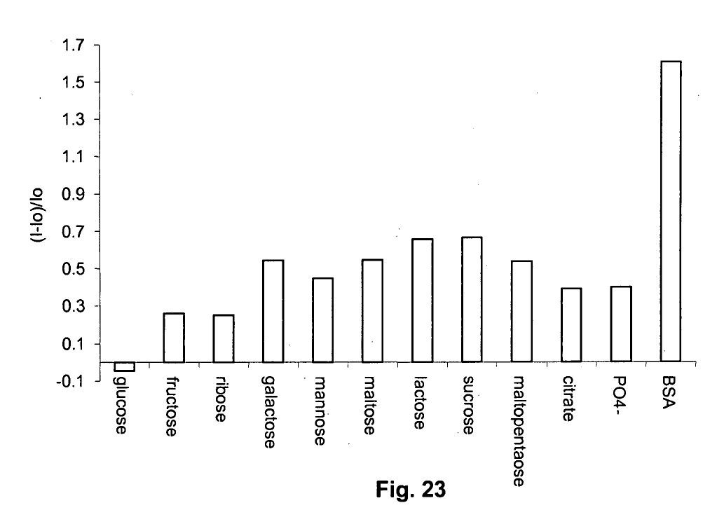

FIG. 23 depicts relative fluorescence intensity changes for solutions of the structure shown in FIG. 19, wherein each R═H, Ln=La³⁺ (5.5×10⁻⁶ M), and X═—OCH₃, in HEPES buffer solution (pH 7.0), for sugar solutions containing various monosaccharides, oligosaccharides, anions (1.1×10⁻³ M), and BSA (1 mg/mL). Excitation was at 360 nm, and fluorescence emission was measured at 404 nm.

### Example 15

Preparation of LPA in MeOH, and of PA in MeOH. Separate aliquots of LPA and PA (1.1×10⁻³ M) were sonicated in MeOH for 5 min. Compound 2, dissolved in MeOH, was added to each these solutions, to a final concentration of 5.53×10⁻⁶ M.

### Example 16

Preparation of LPA in plasma samples. Aliquots of LPA in distilled water were added to lyophilized commercial blood plasma samples via microsyringe. A sufficient volume of a MeOH solution of LaCl₃ (1×10⁻³ M) was added to the LPA/plasma mixture to achieve the original dilution of the solid components. This suspension was mixed and sonicated for 5 min. The resulting mixture was filtered through a pre-column HPLC filter. A solution of Compound 2 in MeOH was added to the filtered plasma samples to a final concentration of Compound 2 of 5.53×10⁻⁶ M. An otherwise identical control solution was prepared from the plasma extract and MeOH solution of Compound 2, but with no LPA. Fluorescence spectra of both solutions were then measured.

### Example 17

Preparation of gangliosides. Aliquots of the gangliosides were dissolved in 0.1 M HEPES buffer, pH 7.0, to a final ganglioside concentration of 0.5 mg/mL. Solutions of the other analytes used for comparison (and for interference testing) were prepared by dissolving the analytes in HEPES buffer to a final concentration of each analyte of 1.1×10⁻³ M. Compound 2 in MeOH was added to each sample to a final concentration of 5.53×10⁻⁶ M. “Blank” samples for comparison testing were prepared with the buffer containing Compound 2, but without analyte.

### Example 18

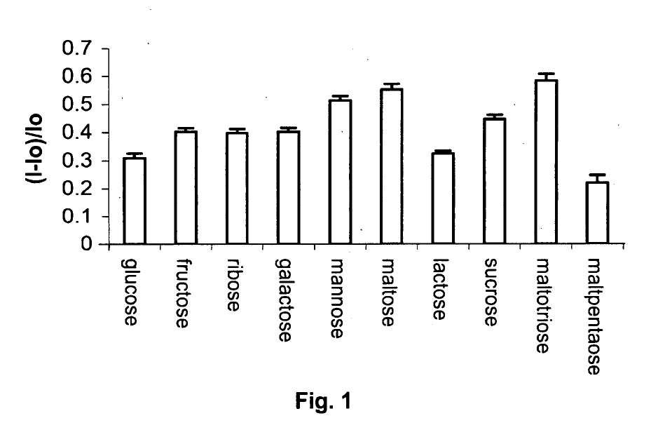

A substantial fluorescence increase was observed when saccharides were mixed with Compound 1 in neutral buffer. Neutral saccharides (1.1×10⁻³ M) were added to 0.1. M HEPES in water, pH 7.0, containing 5.53×10⁻⁶ M of Compound 1. Fluorescence was measured at excitation λₑₓ=360 nm, and emission λₑₘ=400 nm. See FIG. 1, which depicts relative fluorescence intensity changes for several saccharides under these conditions. The standard deviation in relative fluorescence intensity ranged from 0.01-0.027 (n=3 for each saccharide).

### Example 19

We also observed that lanthanum-containing Compound 1 exhibited high selectivity for neutral sugars as compared to several potentially interfering agents. For example, we found that glycerol, phosphates, proteins, citrate, and hydroxy-acids such as sialic acid did not induce appreciable fluorescence enhancement in solutions of Compound 1 (data not shown).

### Example 20

Solutions of La-containing Compound 1 (5.53×10⁻⁶ M, λₑₓ 360 nm, λₑₘ 400 nm, 0.1 M HEPES, pH 7.0) exhibited enhanced fluorescence in the presence of both the monosialoganglioside GM1 and its neutral asialo analog, asialo-GM1 (0.5 mg/mL). In fact, the fluorescence signal with asialo-GM1 was stronger than that from sialic acid-containing GM1 (data not shown).

### Example 21

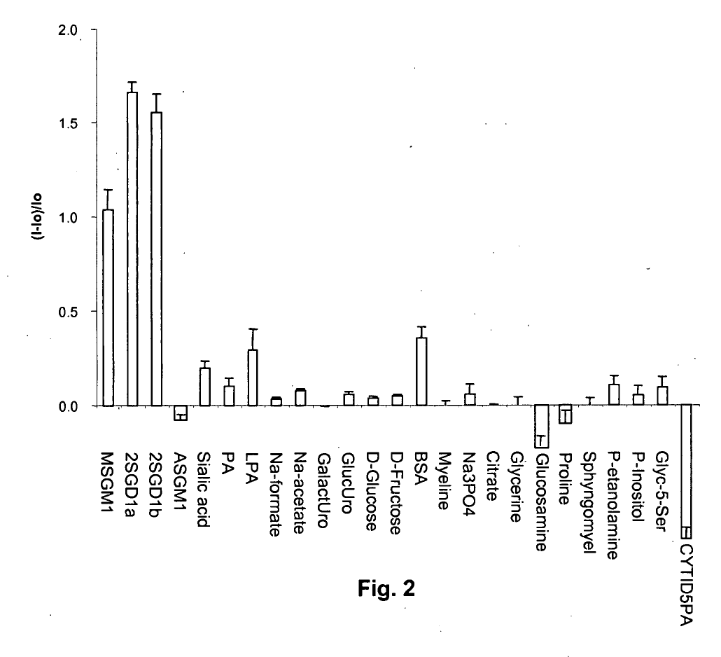

The Eu³⁺-containing Compound 2 showed no substantial change in fluorescence emission in the presence of neutral fructose, glucose, or asialo-GM1 in buffer solution. However, fluorescence increased substantially in the presence of sialic acid-containing gangliosides. See FIG. 2, which depicts the relative fluorescence intensity changes of solutions of Compound 2 (5.53×10⁻⁶ M) in HEPES buffer, pH 7.0, in the presence of various gangliosides, phospholipids (LPA and PA), and other charged and neutral analytes. Ganglioside concentration=0.5 mg/mL. Concentration of other analytes=1.1×10⁻³ M. Asialoganglioside=ASGM1; monosialoganglioside=GM1; disialogangliosides=GD1a and GD1b. The standard deviations (n=3) of the relative fluorescence intensity for each analyte ranged from 0.01-0.11.

### Example 22

Relatively much weaker emission changes were observed with uronic acids and simple carboxylates. The disialoganglioside—Compound 2 solutions showed stronger emission than the monosialo GM1—Compound 2 solutions.

### Example 23

Saccharides (1.1×10⁻³ M) added to Compound 1 (5.53×10⁻⁶ M in H₂O, with 0.1 M HEPES, pH 7.0) were readily monitored by increases in fluorescence emission (FIGS. 1 and 2). Without wishing to be bound by this hypothesis, we believe that lanthanide coordination to salophenes brings the ligand into a more rigid cyclic structure, thereby increasing ligand-centered fluorescence emission. In the ¹H NMR of a solution of Compound 1 and D-glucose in D₂O, the imine protons of Compound 1 exhibited a modest upfield shift, as has also been seen for other salophene-metal complexes (data not shown).

The so-called “continuous variation” method has been used to determine the stoichiometry of the complexes between sugars and Compound 1. Without wishing to be bound by this hypothesis, our results suggested that a 1:1 stoichiometry between glucose, maltose, or maltotriose, on the one hand, and Compound 1, on the other hand, was formed. Glucose, maltose, and maltotriose exhibited binding constants of 500, 1666, and 2500 M⁻¹ respectively to Compound 1. These values compared favorably to those that have been reported for sugar-boronate complexes, which have been the current reagents of choice for sugar detection in aqueous and mixed-aqueous media. Fluorescence emission increased in the presence of neutral sugars by about 25% to about 60%, even at sugar concentrations ˜10⁻⁵ M.

### Example 24

Common anions, including citrate, phosphate, and pyrophosphate, produced relatively weaker emission changes with Compound 1 under otherwise similar reaction conditions. Bovine serum albumin-containing solutions exhibited increased fluorescence only when glucose was present. (data not shown)

### Example 25

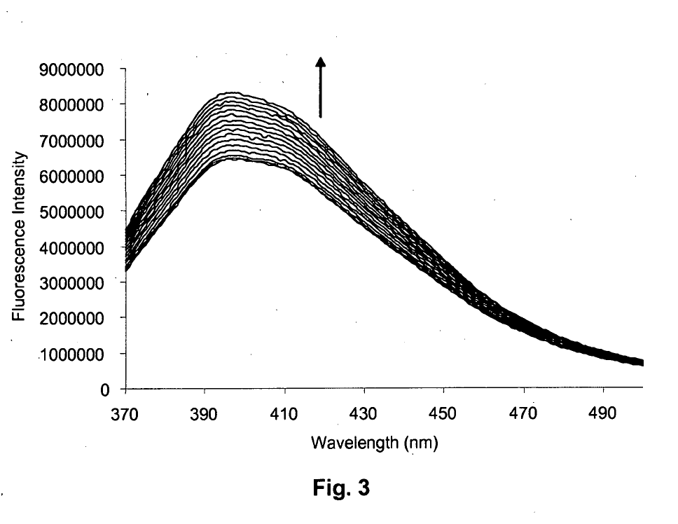

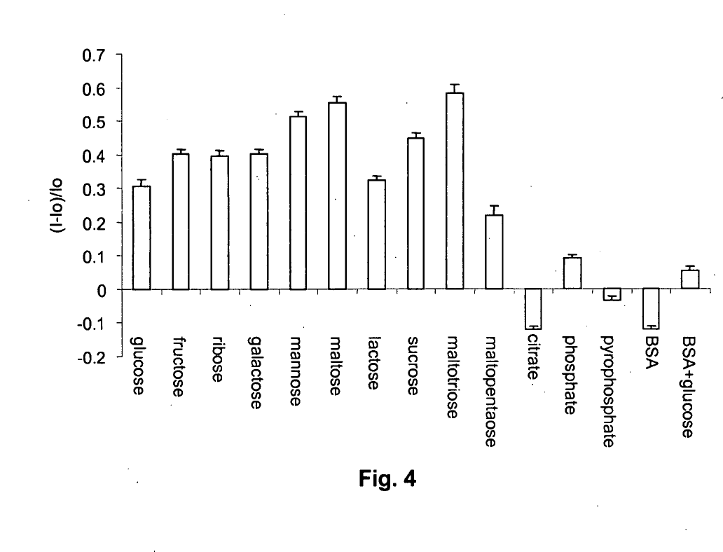

FIG. 3 depicts fluorescence emission for mixtures of Compound 1 with D-glucose at various concentrations. In all cases, the concentration of Compound 1 was 6×10⁻⁶ M in 0.1M HEPES buffer, pH 7.0, and the excitation frequency was 360 nm. The several curves correspond to different concentrations of D-glucose, from zero on the lowest curve (i.e., Compound 1 alone), to a D-glucose concentration of 6×10⁻⁴M for the top curve.

### Example 26

FIG. 4 depicts the changes observed in relative fluorescence emissions (at 400 nm) for solutions containing various monosaccharides or oligosaccharides, anions (1.1×10⁻³ M), BSA (1 mg/mL), or a mixture of BSA and glucose (1 mg/mL and 1.1×10⁻³ M, respectively), on the one hand; with Compound 1, on the other hand, at a concentration of 5.53×10⁻⁶ M in HEPES buffer solution (pH 7.0). The standard deviations (n=3 for each analyte) of the relative fluorescence intensities ranged from 0.01-0.027.

### Example 27

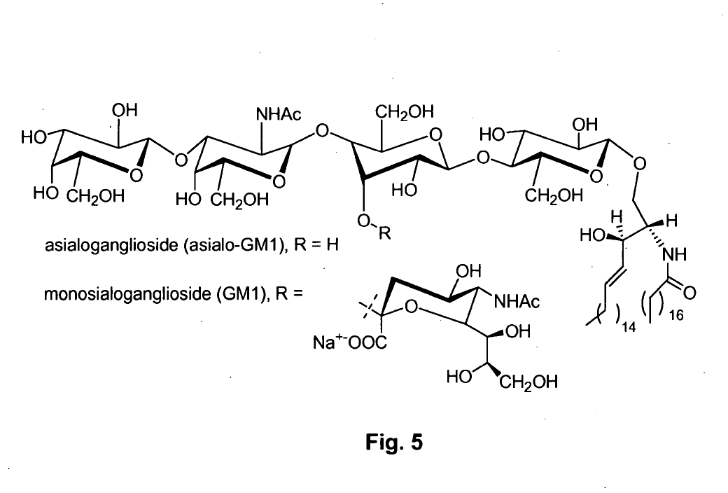

Selective detection of gangliosides under neutral conditions. FIG. 5 depicts the structures of asialo-GM1 and GM1. An increase or decrease in total sialic acid levels in biological fluids (conjugated plus freely circulating sialic acid) can indicate the occurrence of certain cancers. One embodiment of the present invention provides improved sensing agents and methods for determining sialic acid-containing gangliosides.

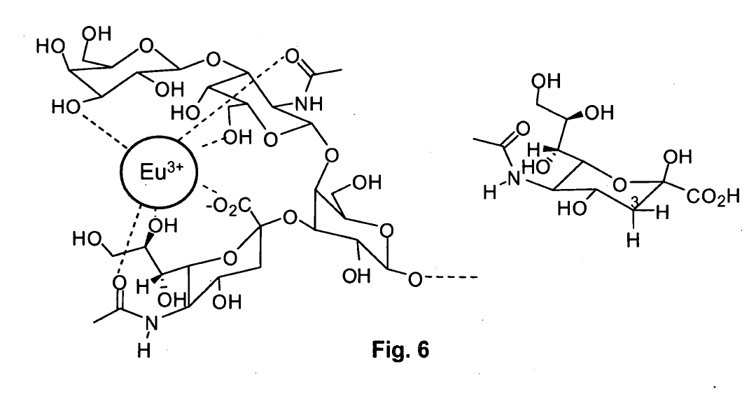

Selectivity towards various anionic substrates can be tuned via the choice of lanthanide metal center. In general, with a higher atomic number within the lanthanide series (i.e., towards the right in the periodic table), the atomic radius decreases, and selectivity for anionic substrates is enhanced. Affinity towards anionic substrates is also enhanced by employing metal atoms with a +4 or higher charge, rather than a +3 charge (e.g., Ce⁴⁺, Th⁴⁺, Pa⁴⁺, U⁴⁺, Zr⁴⁺). It is believed that this is the first report of selective fluorescence detection of asialo-GM1 or GM1 using a composition containing Eu³⁺. The higher affinity of Eu³⁺ towards GM1 than to sialic acid may be due not only to an electrostatic interaction with the GM1 sialic acid carboxylate, but also to secondary interactions with the proximal oligosaccharide hydroxyls, although we do not wish to be bound by this hypothesis. If this hypothesis is correct, then this interaction should result in a coordination shell about Eu⁺³ as depicted in FIG. 6, the left half of which depicts the coordination of GM1 to Eu³⁺, and the right half of which depicts free sialic acid. (FIG. 6 is adapted in part from L. Sillerud et al., Biochemistry vol. 17, pp. 2619 ff (1978).)

Thus we hypothesize that Compound 2 may afford enhanced signaling when charged gangliosides are present, as compared to solutions containing Compound 2 and only neutral sugars and sialic acid.

### Example 28

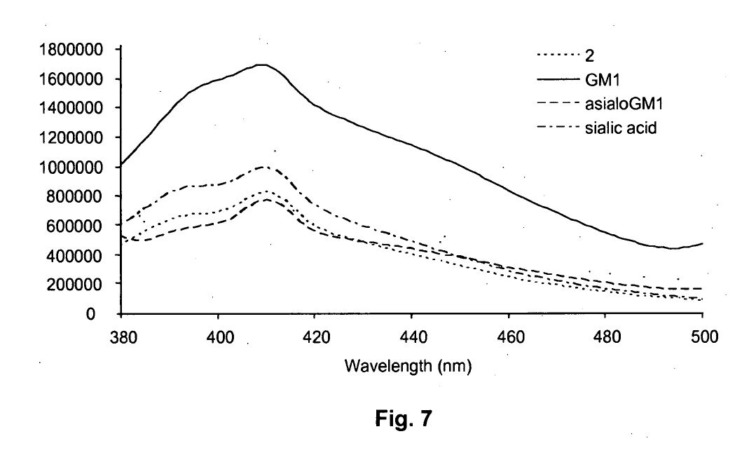

Compound 2 also appears to be more sensitive for the detection of sialic acid-containing gangliosides when compared to the detection of asialo GM1, as depicted in FIG. 7. FIG. 7 depicts the fluorescence intensity (following excitation at 360 nm) at various emission wavelengths for solutions of Compound 2 (5.53×10⁻⁶ M), both alone and in combination with gangliosides (1.1×10⁻⁴ M) or sialic acid (1×10⁻³ M) in 0.1 M HEPES buffer solution (pH 7.0).

### Example 29

By contrast, Compound 1 afforded greater fluorescence enhancement in the presence of neutral asialo GM1 (data not shown). It appears that the smaller the ionic radius of the lanthanide is, the stronger are its ligand interactions, although we do not wish to be bound by this hypothesis. The salophene ligands of Compounds 1 and 2 contain both polar and nonpolar moieties, which assists in binding the polar and nonpolar groups of the analyte. The combination of these structural features, along with the smaller ionic radius of Eu³⁺ as compared to La³⁺, apparently renders Compound 2 better at detecting anionic gangliosides than Compound 1.

Without wishing to be bound by this hypothesis, we believe that the sialic acid residue of GM1 binds Eu⁺³ via multiple coordination sites, as depicted in FIG. 6. Free sialic acid binding (predominantly the β-pyranose form) can bind metal atoms, through metal ion coordination with the carboxylate, pyranose ring, and glycerol side-chain oxygens of sialic acid. However, when sialic acid was titrated with Compound 2 in D₂O, the ¹H NMR signals corresponding to the protons on the glycerol side-chains and protons on the pyranose ring underwent substantial peak-broadening. The 3Hₐₓ proton, on the same side of the pyranose as the carboxylate moiety, is relatively closer to the metal site than the 3-H_{eq} proton. The axial proton resonance on carbon atom 3 broadens more than that of 3-H_{eq}.

### Example 30

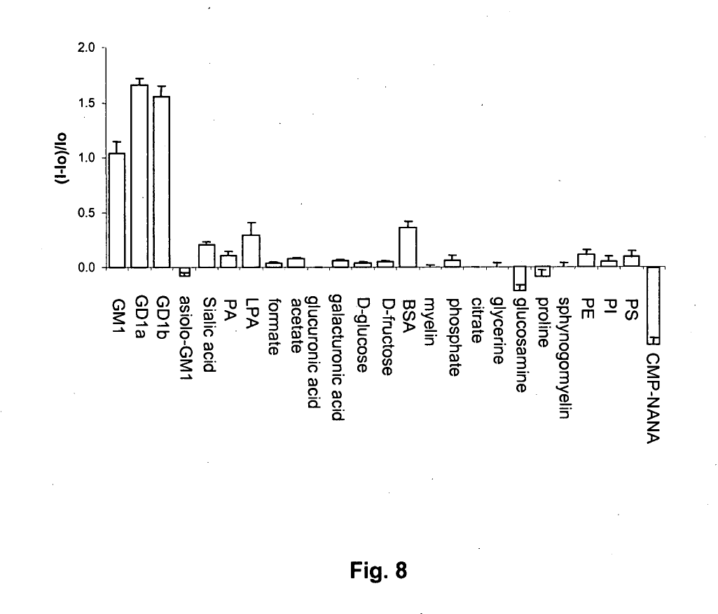

FIG. 8 depicts relative fluorescence intensities for solutions of compound 2 (5.53×10⁻⁶ M) in HEPES buffer pH 7.0 with various gangliosides, phospholipids, and other charged and neutral analytes. The ganglioside concentrations were 0.5 mg/mL each (ca. 10⁻⁴ M); the concentrations of proteins, such as myelin and BSA, were 1 mg/mL; and the concentrations of other analytes were each 1.1×10⁻³ M. The standard deviations (n=3) of the relative fluorescence intensities for the analytes ranged from 0.01-0.11. Asialoganglioside GM1=Asialo-GM1; monosialoganglioside GM1=GM1; disialogangliosides=GD1a and GD1b; L-α-phosphatidylinositol=Pl; L-α-phosphatidylethanolamine=PE; L-α-phosphatidylserine=PS; CMP-NANA=Cytidine-5′-monophospho-N-acetylneuraminic acid.

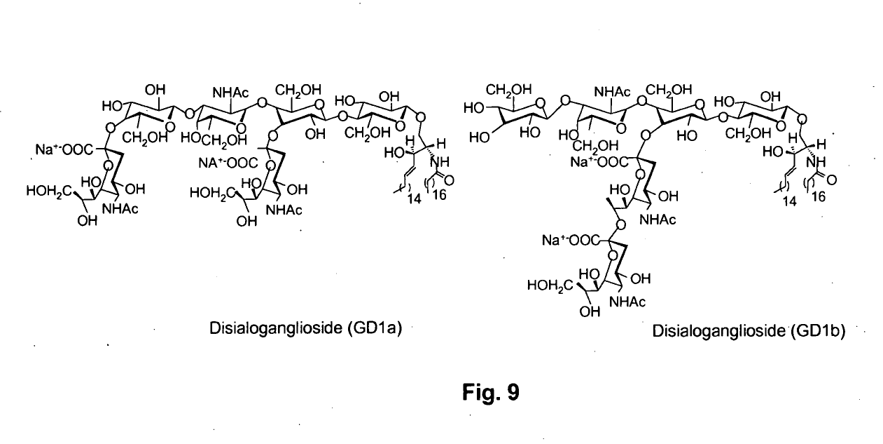

Many compounds are present in typical ganglioside extracts from biological sources. Other typical components include free sialic acid, phospholipids, myelins, proline, and glucosamine. These and other structurally-related compounds did not substantially interfere with ganglioside detection in neutral buffer solution. See FIG. 8. Interestingly, complexes of the disialogangliosides GD1a or GD1b with Compound 2 showed stronger fluorescence emission than did the corresponding complex of monosialo GM1 with Compound 2. See FIG. 9, which depicts the structures of disialogangliosides GD1a and GD1b.

Although not wishing to be bound by this hypothesis, these results suggested that affinity towards Compound 2 is enhanced by a sialic acid moiety bound to an oligosaccharide. Comparison of the fluorescence spectra of Compound 2 in the presence of GM1, in the presence of neutral asialo GM1, and in the presence of several other analytes suggested that proximal oligosaccharide-sialic acid groups substantially enhanced signal transduction. See FIG. 8.

### Example 31

Tetracycline is a tetradentate molecule that may also be used with a metal atom center in practicing an alternative embodiment of the present invention.

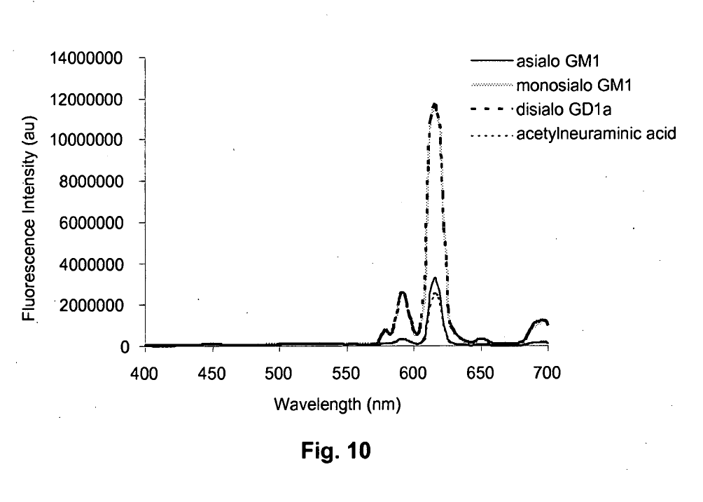

Compounds 1 and 2 were both found to be more selective than the europium(III)-tetracycline (Eu—Tc) complex, however, in detecting gangliosides. FIG. 10 depicts fluorescence intensity versus wavelength for several Eu—Tc complexes (5.53×10⁻⁶ M) with gangliosides (1.1×10⁻⁴ M) or sialic acid (1×10⁻³ M) in 0.1 M HEPES buffer solution (pH 7.0). The excitation frequency was 390 nm.

### Example 32

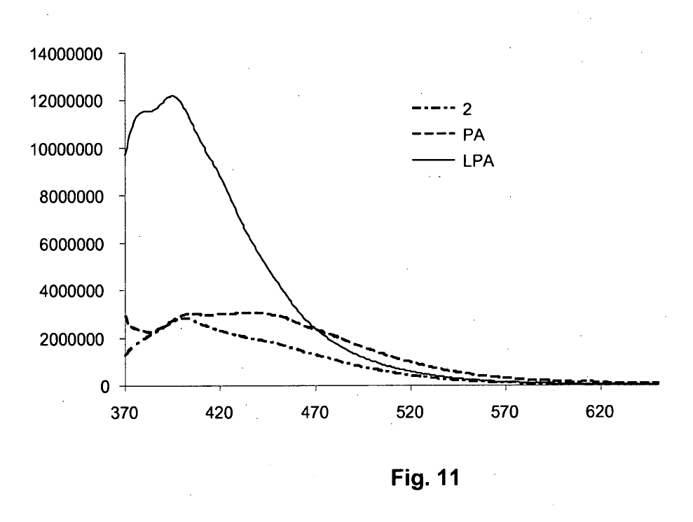

Selective detection of lysophosphatidic acid. The affinity of Compound 2 towards amphiphilic analytes appears to be solvent-dependent. Selectivity for specific phospholipids can be achieved in MeOH. The phospholipids lysophosphatidic acid (LPA) and phosphatidic acid (PA) are soluble in aqueous media. However, they are only sparingly soluble in MeOH. However, LPA and PA can be solubilized via sonication in the presence of Compound 2 in MeOH. FIG. 11 depicts fluorescence intensity versus wavelength for Compound 2 and solutions of Compound 2 (5.53×10⁻⁶ M), with either LPA or PA (1.1×10⁻⁴ M) in MeOH. The excitation frequency was 360 nm.

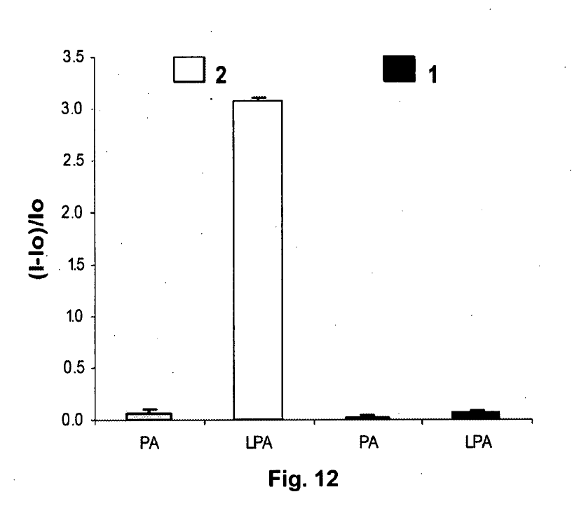

FIG. 12 depicts relative fluorescence intensity changes for solutions of Compound 1 or 2 (5.53×10⁻⁶ M) with LPA or PA (1.1×10⁻⁴ M) in MeOH. The excitation frequency was 360 nm, and emission was measured at 400 nm. The standard deviations (n=3) of the relative fluorescence intensities for the analytes ranged from 0.01-0.03.

MeOH solutions containing Compound 2 exhibited increased fluorescence emission in the presence of commercially-purchased LPA (oleoyl-L-α-lysophosphatidic acid Na salt, 5.53×10⁻⁶ M, λₑₓ 360 nm, λₑₘ 403 nm). By contrast, solutions containing commercial PA (3-sn-phosphatidic acid Na salt) exhibited only minor fluorescence changes at 400 nm (FIGS. 11 and 12), even at millimolar PA levels.

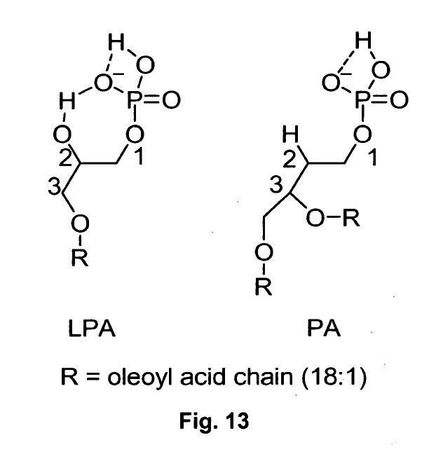

Without wishing to be bound by this hypothesis, the differing affinities of LPA and PA for Compound 2 may be attributed to the presence or absence of intramolecular hydrogen bonding to the respective phosphate moieties. Intramolecular hydrogen bonding between the phosphate and the 2-sn-OH moieties has been observed in the crystal structure of LPA, and is believed to persist under physiological conditions. See, e.g., E. Kooijman et al., Biochemistry, vol. 44, pp. 17007 ff (2005). By contrast, a homologous —OH group is not available for hydrogen bonding in PA. See FIG. 13, which depicts intramolecular hydrogen bonding patterns of LPA and PA that we hypothesize explain the lower pKa of LPA. The phosphate hydroxyl of LPA is more prone to ionize than is that of PA. This tendency generates a higher negative charge on the LPA phosphate, which we hypothesize leads to enhanced binding to Compound 2, dominated by ionic interactions.

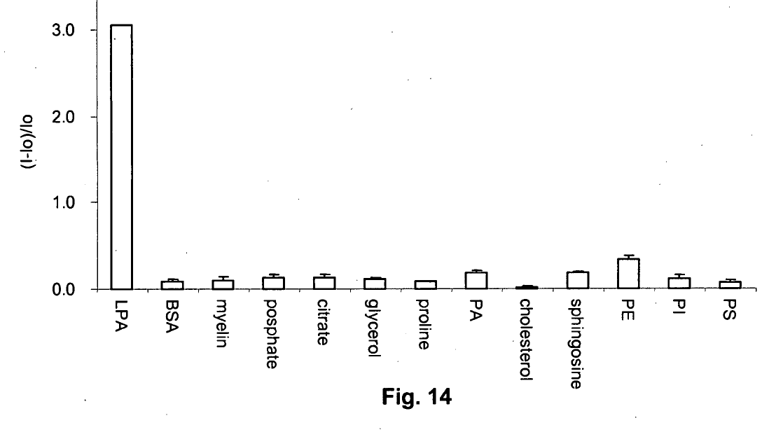

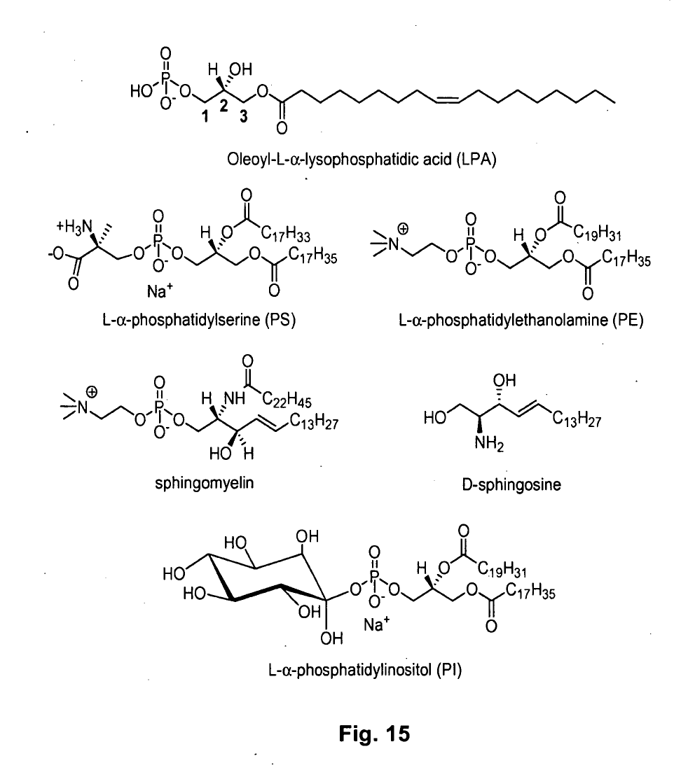

Without wishing to be bound by this hypothesis, it appears that the free hydroxyl oxygen of LPA may also serve as a coordination binding site for the lanthanide metal atom. A second coordinating site, especially one containing a hard atom such as oxygen or nitrogen, can enhance lanthanide affinity, especially in aqueous media. Compare FIG. 6. Indeed, we observed significant broadening only of the ¹H NMR resonances corresponding to protons on carbons 1-3 of LPA. The NMR broadening indicates that the phosphorus of LPA is close to the metal center, and provides evidence of binding. We hypothesize that the availability of the hydroxyl oxygen coordination site, together with the relatively higher negative charge of LPA as compared to PA, enhance Compound 2's selectivity for LPA over PA. FIG. 14 depicts relative fluorescence intensity changes of solutions of Compound 2 in MeOH (5.53×10⁻⁶ M), in the presence of phospholipid LPA, or PA (ca. 10⁻³ M), or other charged and neutral analytes. The concentration of each of the other analytes was 1.1×10⁻³ M. The standard deviations (n=3) of the relative fluorescence intensities for each analyte ranged from 0.01-0.11. FIG. 15 depicts the structures of LPA and the other phospholipids tested in these experiments.

### Example 33

Detection of Ovarian Cancer and Other Gynecological Cancers. Each year ovarian cancer kills thousands of women, over 15,000 per year in the United States alone. A principal reason for the low survival rate is the fact there has been no reliable method for early detection. Lysophosphatidic acids (1-acyl-glycerol-3-phosphates), which are simple phospholipids, are markers for the early detection of ovarian cancer. However, current assays for LPA are not well-suited for routine diagnostic and point-of-care use. LPA has been relatively difficult to detect using prior analytical techniques. One aspect of the present invention provides a novel means of detecting LPA selectively, using Compound 2, or one of the other compounds depicted in FIGS. 18 and 19, to complex LPA and thereby to increase its fluorescence in solvents such as MeOH. Another aspect of the present invention uses Compound 2 to detect ovarian cancer and other gynecological cancers by determining LPA in a sample taken from a patient, for example in circulating plasma. The data shown in FIG. 14 demonstrate that common components of phospholipid extracts should not substantially interfere with fluorescent detection of LPA with Compound 2 in MeOH solution.

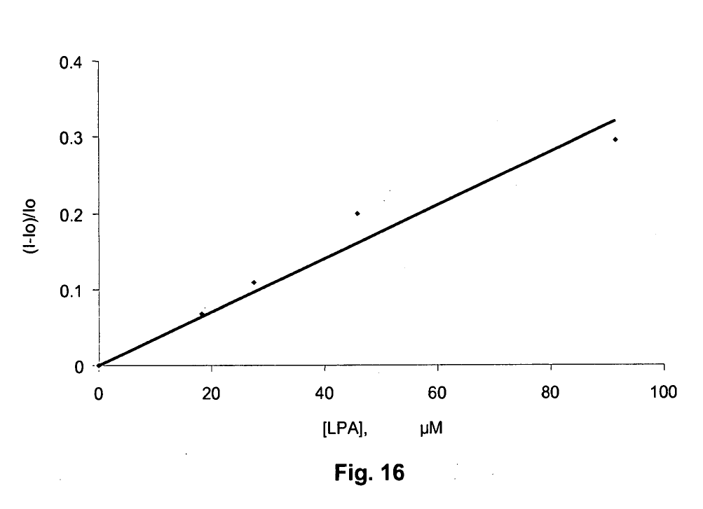

We have observed a strong correlation between LPA concentration and fluorescence intensity in MeOH extracts of lyophilized human plasma that had been spiked with LPA. LaCl₃ was also added to the mixture, to bind neutral sugar compounds, and thereby remove some potentially interfering neutral components. See FIG. 16, which depicts relative fluorescence emission versus concentration of LPA in methanolic extracts of blood plasma samples containing Compound 2; with excitation at 360 nm, and emission measured at 437 nm. When carried out in triplicate the standard deviation of the relative fluorescence intensity did not exceed 0.03. LPA was successfully detected over at least the concentration range 1.83×10⁻⁵ M to 9.15×10⁻⁵ M. Y. Xu et al. (1998) reported an LPA concentration range in plasma from healthy control patients from below 0.1 to 6.3 μM, with a mean of 0.6 μM; while the concentration in patients with ovarian cancer was between 1 and 43.1 μM, with a mean of 8.6 μM. (Since the novel method may be used to detect LPA in methanol, a plasma sample may first be lyophilized and then reconstituted in methanol to make the sample more concentrated; thus this range of concentrations may be detected with our current method.) Plasma LPA levels are an important marker for ovarian cancer, and possibly other gynecological cancers as well. However, existing means of detecting plasma LPA are not well-suited for routine clinical diagnosis. The present invention provides a convenient, easily implemented means for determining plasma LPA levels, facilitating the early diagnosis of ovarian cancers, and possibly other gynecological cancers as well.

The complete disclosures of all references cited in this specification are hereby incorporated by reference. In the event of an otherwise irreconcilable conflict, however, the present specification shall control.

What is claimed:

1. A process for detecting a lysophosphatidic acid in a sample, said process comprising mixing the sample with a composition comprising 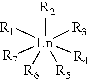
and observing the extent, if any, to which the mixture displays altered fluorescence emission at wavelengths characteristic of binding to a lysophosphatidic acid; wherein:
(a) Ln is a metal atom having atomic number from 58 (Ce) to 71 (Lu)
(b) R₁, R₂, R₃, R₄ R₅, R₆, and R₇ are a plurality of ligands other than solvent molecules, coordinated to the Ln metal atom; wherein there are from 2 to 7 such ligands R₁, R₂, R₃, R₄ R₅, R₆, and R₇ per Ln metal atom, and from 0 to 5 of the potential ligands R₁, R₂, R₃, R₄ R₅, R₆, and R₇ may be empty; wherein the ligands R₁, R₂, R₃, R₄ R₅, R₆, and R₇ may be the same or different; and wherein the ligands R₁, R₂, R₃, R₄ R₅, R₆, and R₇ may be monodentate or polydentate;
(c) the plurality of ligands R₁, R₂, R₃, R₄ R₅, R₆, and R₇, taken as a group, possess the following characteristics:
(i) there are a plurality of P, S, O, or N atoms available for coordinating to the Ln metal atom;
(ii) there are a plurality of hydrophilic groups, sufficient to impart water solubility to the composition;
(iii) there are both polar and nonpolar groups, sufficient to promote binding both to the polar moieties and to the nonpolar moieties of a lysophosphatidic acid, respectively;
(iv) the conformation of the composition, and the distribution of the polar and nonpolar groups promote binding between the Ln metal atom and the phosphate group of a lysophosphatidic acid.

2. A process as recited in claim 1, wherein at least one of the nonpolar groups comprises an aromatic ring.

3. A process for diagnosing gynecological cancer in a patient, comprising assaying a sample from the patient for lysophosphatidic acid levels by the process of claim 1; wherein elevated lysophosphatidic acid levels indicate an elevated likelihood of gynecological cancer in the patient.

4. A process as recited in claim 3 for diagnosing ovarian cancer in a patient; wherein lysophosphatidic acid elevated levels indicate an elevated likelihood of ovarian cancer in the patient.

5. A process as recited in claim 3, wherein the sample comprises plasma, and wherein the composition comprises a methanolic solution of the following compound:

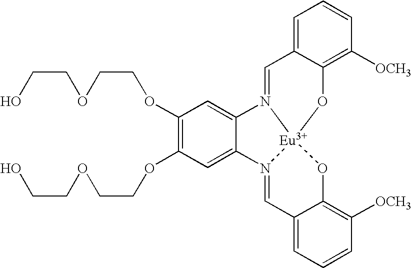

6. A process for detecting one or more neutral sugars in a sample, said process comprising mixing the sample with a composition comprising 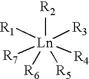
and observing the extent, if any, to which the mixture displays altered fluorescence emission at wavelengths characteristic of binding to a neutral sugar; wherein:
(a) Ln is a metal atom selected from the group consisting of the lanthanides, the actinides, the transition metals, Sc, Y, Ca, Sr, Ba, and Ra.
(b) R₁, R₂, R₃, R₄ R₅, R₆, and R₇ are a plurality of ligands other than solvent molecules, coordinated to the Ln metal atom; wherein there are from 2 to 7 such ligands R₁, R₂, R₃, R₄ R₅, R₆, and R₇ per Ln metal atom, and from 0 to 5 of the potential ligands R₁, R₂, R₃, R₄ R₅, R₆, and R₇ may be empty; wherein the ligands R₁, R₂, R₃, R₄ R₅, R₆, and R₇ may be the same or different; and wherein the ligands R₁, R₂, R₃, R₄ R₅, R₆, and R₇ may be monodentate or polydentate;
(c) the plurality of ligands R₁, R₂, R₃, R₄ R₅, R₆, and R₇, taken as a group, possess the following characteristics:
(i) there are a plurality of P, S, O, or N atoms available for coordinating to the Ln metal atom;
(ii) there are a plurality of hydrophilic groups, sufficient to impart water solubility to the composition;
(iii) there are both polar and nonpolar groups, sufficient to promote binding both to the polar moieties and to the nonpolar moieties of the neutral sugar, respectively.

7. A process as recited in claim 6, wherein at least one of the nonpolar groups comprises an aromatic ring.

8. A process for detecting one or more gangliosides in a sample, said process comprising mixing the sample with a composition comprising 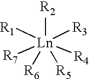
and observing the extent, if any, to which the mixture displays altered fluorescence emission at wavelengths characteristic of binding to a ganglioside; wherein:
(a) Ln is a metal atom selected from the group consisting of the lanthanides, the actinides, the transition metals, Sc, Y, Ca, Sr, Ba, and Ra.
(b) R₁, R₂, R₃, R₄ R₅, R₆, and R₇ are a plurality of ligands other than solvent molecules, coordinated to the Ln metal atom; wherein there are from 2 to 7 such ligands R₁, R₂, R₃, R₄ R₅, R₆, and R₇ per Ln metal atom, and from 0 to 5 of the potential ligands R₁, R₂, R₃, R₄ R₅, R₆, and R₇ may be empty; wherein the ligands R₁, R₂, R₃, R₄ R₅, R₆, and R₇ may be the same or different; and wherein the ligands R₁, R₂, R₃, R₄ R₅, R₆, and R₇ may be monodentate or polydentate;
(c) the plurality of ligands R₁, R₂, R₃, R₄ R₅, R₆, and R₇, taken as a group, possess the following characteristics:
(i) there are a plurality of P, S, O, or N atoms available for coordinating to the Ln metal atom;
(ii) there are a plurality of hydrophilic groups, sufficient to impart water solubility to the composition;
(iii) there are both polar and nonpolar groups, sufficient to promote binding both to the polar moieties and to the nonpolar moieties of the ganglioside, respectively.

9. A process as recited in claim 8, wherein at least one of the nonpolar groups comprises an aromatic ring.

10. A process for detecting sialic acid in a sample, said process comprising mixing the sample with a composition comprising 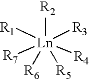
and observing the extent, if any, to which the mixture displays altered fluorescence emission at wavelengths characteristic of binding to sialic acid; wherein:
(a) Ln is a metal atom selected from the group consisting of the lanthanides, the actinides, the transition metals, Sc, Y, Ca, Sr, Ba, and Ra.
(b) R₁, R₂, R₃, R₄ R₅, R₆, and R₇ are a plurality of ligands other than solvent molecules, coordinated to the Ln metal atom; wherein there are from 2 to 7 such ligands R₁, R₂, R₃, R₄ R₅, R₆, and R₇ per Ln metal atom, and from 0 to 5 of the potential ligands R₁, R₂, R₃, R₄ R₅, R₆, and R₇ may be empty; wherein the ligands R₁, R₂, R₃, R₄ R₅, R₆, and R₇ may be the same or different; and wherein the ligands R₁, R₂, R₃, R₄ R₅, R₆, and R₇ may be monodentate or polydentate;
(c) the plurality of ligands R₁, R₂, R₃, R₄ R₅, R₆, and R₇, taken as a group, possess the following characteristics:
(i) there are a plurality of P, S, O, or N atoms available for coordinating to the Ln metal atom;
(ii) there are a plurality of hydrophilic groups, sufficient to impart water solubility to the composition;
(iii) there are both polar and nonpolar groups, sufficient to promote binding both to the polar moieties and to the nonpolar moieties of the sialic acid, respectively.

11. A process as recited in claim 10, wherein at least one of the nonpolar groups comprises an aromatic ring.

12. A process for diagnosing cancer in a patient, comprising assaying a sample from the patient for sialic acid levels by the process of claim 10; wherein abnormal levels of sialic acid indicate an elevated likelihood of cancer in the patient.

13. A composition of matter comprising 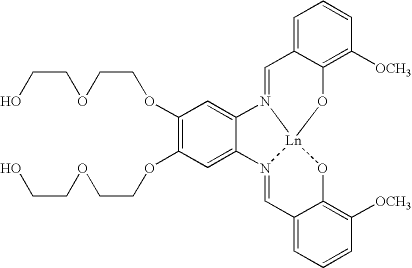
wherein Ln is La³⁺ or Eu³⁺.

14. A composition of matter comprising 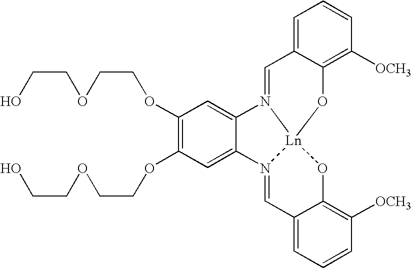
wherein Ln is selected from the group consisting of the lanthanides, the actinides, the transition metals, Sc, Y, Ca, Sr, Ba, and Ra.

15. The composition of claim 10, wherein Ln is La.

16. The composition of claim 10, wherein Ln is Eu.

17. A composition of matter comprising 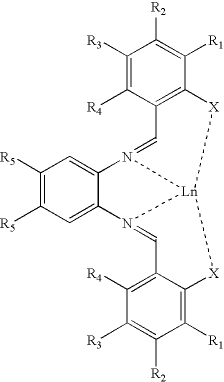
wherein Ln is a metal atom selected from the group consisting of the lanthanides, the actinides, the transition metals, Sc, Y, Ca, Sr, Ba, and Ra; wherein R₁, R₂, R₃, R₄, and R₅ may be the same or different; wherein R₁, R₂, R₃, R₄, and R₅ are independently selected from the group consisting of hydrogen, halogens, groups with heteroatoms (P, O, S, N), saturated or unsaturated C₁ to C₄ aliphatic groups, aromatic groups, glycol, polyethyleneglycol, phosphate, sulphate, and carboxylate; wherein the two X moieties may be the same or different; and wherein each X denotes a halogen atom (F, Cl, Br, I, At), a group V A nonmetal (N, P, As, Sb), or a group VI A nonmetal (O, S, Se, Te, Po).
